(19) United States
(12) Patent Application Publication
(10) Pub. No.: US 2004/0258674 A1
(43) Pub. Date: Dec. 23, 2004

(54) Quercetin supplementation to treat hypertenstion

(76) Inventor: Thunder Jalili, Park City, UT (US)

Correspondence Address: TRASK BRITT, P.O. BOX 2550, SALT LAKE CITY, UT, 84110, US

(21) Appl. No.: 10/822,568

(22) Filed: Apr. 12, 2004

Related U.S. Application Data
(60) Provisional application No. 60/461,861, filed on Apr. 10, 2003.

(51) Int. Cl. A61K 38/43; A61K 31/7048; A61K 31/714; A61K 31/405; A61K 31/355; A61K 31/015; A61K 31/353; A61K 31/375

(52) U.S. Cl. 424/94.1; 514/27; 514/456; 514/458; 514/474; 514/356; 514/52; 514/350; 514/763; 424/702; 424/617

(57) ABSTRACT

A nutritional supplement for improving cardiovascular health via aiding in preventing, slowing the progression of and/or treating hypertension, the supplement comprising quercetin; and a method for aiding in preventing, slowing the progression of and/or treating hypertension are described.

### CROSS-REFERENCE TO RELATED APPLICATION

This application claims the benefit of U.S. Provisional Application No. 60/461,861, filed Apr. 10, 2003, the contents of which are incorporated by reference herein.

### TECHNICAL FIELD

The invention relates to dietary supplements generally and more particularly to a method of using quercetin to treat hypertension.

### BACKGROUND

High blood pressure is known to cause abnormal growth and remodeling of the heart, known as cardiac hypertrophy. The stimulus for abnormal cardiac growth is an increase in cardiac workload due to high systemic blood pressure that must be overcome during ventricular contraction. Increased cardiac workload activates a number of biochemical pathways in the myocyte that trigger cardiac hypertrophy. Fifty million Americans currently suffer from high blood pressure and are undergoing some amount of cardiac hypertrophy and remodeling. Cardiac hypertrophy is a dangerous condition that increases the risk for arrhythmia, myocardial infarction, and heart failure. If left untreated, cardiac hypertrophy can progress at a gross and cellular level to the point where the cardiovascular architecture will not support normal function and will fail as a mechanical pump. This condition of heart failure is currently affecting 4.6 million Americans. In light of this situation, management of high blood pressure is critical to reducing the risk of cardiac hypertrophy and cardiac failure. A natural alternative that could decrease hypertension presents an opportunity to significantly reduce cardiac hypertrophy and promote cardiovascular health in the 50 million Americans currently suffering from hypertension.

Individuals suffering from hypertension have an increased risk of cardiac arrhythmia, cardiac hypertrophy, myocardial infarction, and heart failure. Chronic elevation of myocardial stress due to pressure overload, as in hypertension or aortic stenosis, causes cardiac muscle to undergo hypertrophy with a resulting increase in myocardial thickness. There is normally little or no change in overall cardiac size, so the wall thickening occurs at the expense of the cavity. One of the most damaging forms of hypertrophy is left ventricular hypertrophy (LVH). The prevalence of left ventricular hypertrophy in the population of borderline hypertensives has been estimated to be 16.6% (Melina et al., 1992). The data on prevalence are consistent, but not identical among studies. Some research which examined the role of hypertension and gender in the prevalence of LVH demonstrated rates of 14% and 20% for males and females in normotensives.

In the case of mitral regurgitation, on the other hand, ventricular volume increases with little or no change in maximum developed pressure; wall thickness does not alter significantly but total myocardial mass increases because of the ventricular enlargement. Except in the case of neonates, increase in mass does not alter the number of myocardial cells; the primary histological changes are intracellular, involving changes in the number and arrangement of the sarcomeres. In chronic cases widespread interstitial fibrosis occurs.

Accordingly, there is great interest in both dietary and pharmacological interventions that can prevent or treat existing hypertension. In addition, there is an enormous current public interest in alternative medicines and supplements. With regard to dietary intervention, consuming both flavonoid and vitamin antioxidants have great promise to prevent or reduce blood pressure in humans and experimental animals (Brody et al., 2002; Aviram et al., 2001; Kobaet al., 1992; and Duarte et al., 2001). Compared to pharmacological agents, dietary supplements can be less expensive and have greater popular appeal. Since the use of alternative medicines has greatly increased among Americans in recent years, flavonoid based anti-hypertensive supplements have great commercial potential.

In recent years there has been strong interest in the role phytonutrients may play in preventing heart disease. Dietary flavonoids have received particular attention since many are strong antioxidants and have been shown to reduce the risk of cardiovascular diseases by reducing blood pressure and slowing atherosclerosis.

Recent studies have demonstrated in animals and humans that antioxidants can decrease blood pressure. Furthermore, a number of in vitro studies have demonstrated that quercetin can inhibit Protein Kinase C (PKC), a family of biochemical signaling molecules implicated in governing cardiac hypertrophy and failure.

What is needed therefore is a natural alternative that could decrease blood pressure, which may simultaneously act upon the biochemical pathways that govern cardiac hypertrophy, to significantly reduce cardiac hypertrophy and remodeling in the 50 million Americans currently suffering from hypertension.

### SUMMARY OF THE INVENTION

The invention provides a nutritional supplement composition comprising quercetin. More particularly it provides a nutritional supplement formulation containing a prophylactically effective amount of quercetin that is specifically dedicated to ameliorating, delaying and/or treating hypertension.

The present invention also provides a method for ameliorating, delaying and/or treating hypertension, which comprises administering the nutritional supplement composition to an individual who is at risk or suffers from hypertension.

### BRIEF DESCRIPTION OF THE DRAWINGS

FIG. 1 illustrates mean carotid arterial pressure in groups of rats fed either control diet or a diet supplemented with quercetin and either having had a sham surgery or abdominal aorta constriction (AAC).

FIG. 2A illustrates the pressure overload produced with abdominal aortic constriction (AAC) surgery in rats. PKC βII translocates to the cellular membrane fraction in the heart and this translocation of PKC βII has been previously identified as a critical mediator of cardiac hypertrophy. Quercetin supplementation prevents cardiac PKC βII translocation, and attenuates cardiac hypertrophy. FIG. 2B demonstrates that the other PKC isoforms in the heart are unchanged by both AAC and quercetin supplementation. AAC=abdominal aortic constriction, SH=sham operated, AACQ=abdominal aortic constriction+quercetin, SHQ=sham operated+quercetin.

FIG. 3 illustrates that quercetin prevents vascular dysfunction. AAC rats typically have impaired endothelial dependent relaxation in their aortas when stimulated with acetylcholine (which stimulates vasodilation). Rats given supplemental quercetin have normal endothelial dependent relaxation under the same acetylcholine dose. AAC=abdominal aortic constriction, SH=sham operated, AACQ=abdominal aortic constriction+quercetin, SHQ=sham operated+quercetin. * p<0.05.

FIG. 4 illustrates that activation of Akt is prevented (FIG. 2A) and ERK1/2 (FIG. 2B) is reduced in rats with abdominal aortic constriction fed supplemental quercetin. It has been previously demonstrated that activation of Akt and ERK1/2results in cardiac hypertrophy. AAC=abdominal aortic constriction, SH=sham operated, AACQ=abdominal aortic constriction+quercetin, SHQ=sham operated+quercetin. *,** p<0.05.

FIG. 5 illustrates a quercetin dependent delay in the onset of hypertension using a genetic model of hypertension. Spontaneously hypertensive rats suffer from steadily increasing blood pressure from 6 weeks of age until peaking at 12-15 weeks of age. SH rat diets were supplemented with 1.5 g/kg chow quercetin (SHRQ), which delayed the normal rise in blood pressure. FIG. 5A illustrates a decreased systolic blood pressure in SH rats fed a diet supplemented with quercetin. FIGS. 5B and 5C illustrate that the diastolic pressure and mean arterial pressure did not show a statistically significant changed. *p<0.05.

### DETAILED DESCRIPTION OF THE INVENTION

The consumption of quercetin is quite low in the United States today, estimated at 25 mg/day. This is due to inadequate consumption of quercetin containing foods, in particular, onion (one of the best source of quercetin) consumption is lower in the United States as compared to other parts of the world.

### Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10” as well as “greater than or equal to 10” is also disclosed.

As used herein “a subject” means an animal, including, but not limited to, a human and other mammals.

In this specification and in the claims which follow, reference will be made to a number of terms which shall be defined to have the following meanings: “Treating” or “treatment” does not mean a complete cure. It means that the symptoms of the underlying disease are reduced, and/or that one or more of the underlying cellular, physiological, or biochemical causes or mechanisms causing the symptoms are reduced or delayed. It is understood that reduced or delayed, as used in this context, means relative to the state of the disease, including the molecular state of the disease, not just the physiological state of the disease.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions.

Thus, if there are a variety of additional steps that can be performed it is understood that each of these additional steps can be performed with any specific embodiment or combination of embodiments of the disclosed methods.

As used herein an “effective amount” means a quantity of quercetin and quercetin glycosides, wherein an average adult would experience the health benefits of the compound(s). The health benefits include the prevention, delay and/or treatment of hypertension and the promotion of cardiovascular health. In addition, a dose may be administered over the course of multiple time periods, such as being administered twice daily (one half the daily dose per administration) or once a day. For example, an effective dosage for humans includes a dosage of about 50 mg/kg body weight, which would translate to about 3750 mg per day for a 75 kg human. An effective amount of quercetin includes between about 100 mg/day (0.1 g/day) and about 50000 mg/day (50 g/day), preferably between about 1000 mg/day and 30000 mg/day, more preferably between about 1000 mg/day and about 15000 mg/day, yet more preferably 1000 mg/day and about 5000 mg/day. In an exemplary embodiment, quercetin is provided to a human subject in a dosage of between 100 mg and 2000 mg/day. Dosage is related to the body mass, health status, age and the desired effect relative to an individual. Therefore, the dosage may be varied according to the administration schedule, body mass, age or the like. The dosages set forth herein are safe even for an adult of low body mass, e.g. a 100 pound adult. No toxic effects at the highest dosage set forth herein are known. However, the dosages set forth herein are preferably administered at the lower dosages for subjects having a smaller body weight and at higher dosages for subjects having a larger body weight.

As used herein “hypertension” means an elevation of arterial blood pressure over the accepted norms for a given age (either primary or secondary) and disease caused by elevated blood pressure, including, left ventricular hypertrophy (LVH), right ventricular hypertrophy (RVH), Biventricular Hypertrophy, ischemia of the myocardium and myocardial infarction. Hypertension is defined as systolic blood pressure of 140 mm Hg and above, diastolic blood pressure of 90 or above, or both. The American Heart Association defines pre-hypertension as systolic blood pressure of 120-139, diastolic pressure of 80-89, or both.

Flavonoids, also known as “phenylchromones,” are naturally occurring, water-soluble compounds which have antioxidant characteristics. Flavonoids are found in a variety of vascular plants, such as vegetables and fruits. As a result, flavonoids are present in beverages such as tea and wine (particularly red wine). In contrast to vascular plants, members of the animal kingdom are unable to synthesize the flavone nucleus and obtain flavonoids as a dietary component. Tea and wine, particularly, red wine, may be used to reduce hypertension by the methods of the invention.

It is believed that quercetin, which exhibits some of the strongest antioxidant effects of the flavonoids and reportedly inhibits oxidation and cyto-toxicity of low density lipoproteins (LDL), may have beneficial health consequences since oxidized low density lipoproteins are reported to contribute to the buildup of fatty substances in the arterial wall (to be atherogenic). Lipid peroxidation, which is caused by free radicals (highly reactive molecules with at least one unpaired electron) can be reduced by the antioxidant activity of flavonoids and quercetin.

Flavonoids are conjugated aromatic compounds having the general structure:

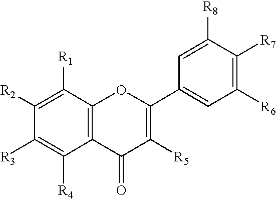

where R₁, R₂, R₃, R₄, R₅, R₆, R₇ and R₈ are independently selected from H and OR′ where R′ is H or an alkyl group having about 1 to 10 carbon atoms (see also, U.S. Pat. No. 6,203,818, incorporated by reference). The most widely occurring flavonoids are flavones and flavonols. The present invention contemplates the use of all flavonoids, however, flavonols and more particularly, quercetin (3,5,7,3′,4′-pentahydroxyflavone), myricetin, (3,5,7,3′,4′,5′,-hexahydroxyflavone), kaempferol (3,5,7,4′-tetrahydroxyflavone), and flavones a\acute{p}igenin (5,7,4′-trihydroxyflavone) and luteolin (5,7,3′,4′-tetrahydroxyflavone) and glycosides thereof are preferred. A preferred flavonoid for use in the invention is quercetin and quercetin glycosides, which are used to illustrate the invention.

Quercetin (3,5,7,3′,4′-pentahydroxyflavone) and quercetin-4-glycoside (hereinafter referred to as quercetin) (Graefe et al., 2001) prevents and/or treats hypertension and reduces blood pressure in established hypertension. Quercetin is a naturally occurring antioxidant found in highest concentrations particularly in onions. It is classified as a flavenoid characterized by 2 benzene rings linked through a heterocyclipyrone ring. It has been reported that quercetin is an antioxidant 10 times more potent than vitamin C (Pedro-Botet et al., 2000).

Structure of Quercetin:

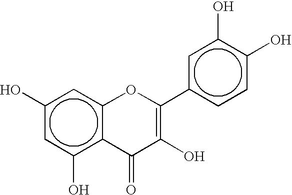

Quercetin acts as an antioxidant that is believed to directly reduce systemic blood pressure and is further believed to act upon the key biochemical pathways responsible for cardiac growth induced by high blood pressure. In one embodiment, a quercetin supplement is prepared as nutritional food, such as a food bar, energy bar, performance snack or performance gel used in preventing and/or treating hypertension. The products produced by POWERBAR® provide non-limiting examples of energy bars, performance snacks and/or energy gels (see also, U.S. Pat. No. 6,248,375, hereby incorporated by reference). In another embodiment, a quercetin supplement is prepared as a liquid beverage, smoothie, diet shake, meal substitute shake, low carbohydrate drink, or health drink. For example, a non-limiting example is a smoothie having quercetin, such as a Jamba Juice® booster. In another embodiment, quercetin is added to a beverage, such as orange juice. In yet another embodiment of the invention a dietary fiber supplement such as oat bran or other natural fiber source may also be added to the composition. In another exemplary embodiment, other supplements such as fish oil or other neutraceutical supplements may be added. A daily supplement containing quercetin may be effective in preventing hypertension in over 50 million people who are at risk for hypertension. With regard to patients with established hypertension, quercetin, a natural agent, may be used to treat this disease. In another exemplary embodiment, a daily supplement containing quercetin may be used to delay the on-set of the hypertension.

The model of using enriched food products to prevent cardiovascular disease currently exists and is economically successful. Three separate margarine products are currently being sold (Benecol®, Take Control®, Smart Balance®). These products have artificially enriched levels of phytosterols, which have been shown to reduce blood cholesterol levels in animal models and clinical studies. Accordingly, these products have been marketed as agents to reduce risk of myocardial infarction.

Hypertensive humans have decreased antioxidant capacity, presumably due to the oxidative stress taxing the body's antioxidant systems (Pedro-Botet et al., 2000). It has been demonstrated in rats that depletion of natural antioxidant systems produced hypertension (Vaziri et al., 2000a; Vaziri et al., 1999). In addition, antioxidant supplementation in hypertensive animal models has been shown to be effective in reducing blood pressure in both spontaneously hypertensive rats, and in rats depleted of their natural antioxidant systems (Newaz and Newal, 1999). Human subjects taking both an anti-hypertensive medication and antioxidant supplements (e.g., Zinc, vitamin E, etc.) have a greater reduction in blood pressure than those on medication alone (Galley et al., 1997). It is believed that antioxidants sequester reactive oxygen species and preserve the levels of nitric oxide, a powerful vasodilator (Vaziri et al., 2000b). Quercetin is believed to be effective in reducing blood pressure in hypertensive models due to its strong antioxidant capacity, documented to be 10 times more potent than vitamin C (Naguib, 2000; Pedro-Botet et al., 2000).

From a variety of models and species, the involvement of PKC in hypertrophy has been demonstrated. Protein Kinase C (PKC) is one of the key signaling pathways implicated in the pathogenesis of cardiac hypertrophy and heart failure. PKC is an enzyme family of serine-threonine kinases in mammalian cells and is thought to activate downstream signaling pathways and genes governing cardiac cell growth. PKC in the heart has been shown to be activated by high blood pressure and to play a role in cardiac hypertrophy in cell culture, conventional animal models, genetically engineered mice, and humans. Mechanical stretch of cardiac myocytes in culture (mimicking hypertension) activates PKC, turns on hypertrophic genes and leads to myocyte hypertrophy. Experimentally induced high blood pressure in rats and guinea pigs also activates PKC and leads to cardiac hypertrophy (Jalili, et al., 1999; Gu and Bishop, 1994). In transgenic mice, cardiac specific overexpression of PKC βII isoform results in cardiac hypertrophy and 100% mortality (Wakasaki et al., 1997). Most importantly, humans in heart failure also have increased PKC activation (Bowling et al., 1999).

Quercetin has been identified as a PKC inhibitor in cell culture. Previous studies have found inhibition of PKC activity in the cytosol and membrane fraction of cells isolated from thyroid (Picq et al., 1989), brain (Ferriola et al., 1989), liver (Mistry et al. 1997), and fibroblasts (Lee and Lin, 1997). These studies all used similar protocols; incubation cells with similar (i.e., μM) concentrations of quercetin. However, there is very little in vivo data examining the effect of quercetin on PKC activity.

There has been one study examining the role of quercetin to prevent cardiac hypertrophy in mice (Wang et al., 1999). An oral, 5 day, 120 mg/kg/day dose of quercetin prevented cardiac hypertrophy in mice with surgically induced abdominal aortic constriction designed to produce pressure overload. The mechanism for this observation has not been identified.

Quercetin, or quercetin glycosides or similar isoforms can serve as an economical natural agent to fight hypertension, and may be combined with one or more other products to further lower the risks of cardiovascular disease. For example, quercetin may be added to well known nutritional supplements and/or non-flavonoid antioxidants, e.g., selenium, vitamin E (tocopherol, particularly α- and δ-tocopherol, etc.), vitamin C (ascorbic acid) and coenzyme Q10 and/or dietary fiber supplements. Quercetin may be added to other flavonoids and flavonols, such as myricetin, (3,5,7,3′,4′,5′,-hexahydroxyflavone), kaempferol (3,5,7,4′-tetrahydroxyflavone), and flavones apigenin (5,7,4′-trihydroxyflavone) and luteolin (5,7,3′,4′-tetrahydroxyflavone) and glycosides thereof.

In addition to quercetin, the supplement of the invention may also contain a vitamin B complex member, such as vitamin B₁₂ and vitamin B₆. Each member of the vitamin B complex group is a distinctly different substance with different functions. Therefore, quercetin may be combined with one or more vitamin B complex member.

Other well known nutritional supplements such as amino acids and derivatives thereof, e.g., L-arginine, non-flavonoid antioxidants, e.g., selenium, vitamin E isoforms (α- or δ-tocopherol, etc.), vitamin C (ascorbic acid), coenzyme Q10, niacin and beta-carotene may be effectively used in the nutritional supplement of this invention.

Other additives may be incorporated in the nutritional supplement of the present invention. Such additives include minerals, e.g., boron, etc. and trace metals such as zinc, magnesium, manganese, chromium, molybdenum, copper, iron, calcium, and potassium; and other micronutrients such as thiamine, riboflavin, niacin, pantothenic acid, pyridoxine, choline, biotin, inositol, para-aminobenzoic acid, vitamin D, vitamin K, vitamin A, etc. In another embodiment of the invention a dietary fiber supplement such as oat bran or other natural fiber source may also be added to the composition. In one embodiment, quercetin is formulated to produce a food or food bar. In another embodiment, quercetin is added to an aqueous based drink.

The nutritional supplement may, where desirable and appropriate, further include a pharmaceutically acceptable carrier such as lactose, glucose, sucrose, corn starch, potato starch, cellulose acetate, ethyl cellulose, etc. Diluents and other additives such as one or more pharmaceutically acceptable binding agents, fillers, supports, thickening agents, taste-improving agents, coloring agents, preservatives, stabilizers, regulators, emulsifiers or mixtures thereof may be used depending on the form of the composition employed.

The supplement is preferably administered orally but may be administered parenterally or topically. Suitable forms for oral, topical or parenteral administration include tablets, capsules, lozenges, syrups, granules, solutions, sub-dermal patch, lotion, and suspensions, which contain unit doses of the supplement for administration once or several times a day; or at various time intervals. The nutritional supplement composition of the invention will typically be administered orally as a tablet, capsule, gel tabs, or liquid. More preferably, the nutritional supplement composition of the invention will typically be administered orally as a food or energy bar or as a nutritional drink. In addition, sustained release formulations can be formulated and prepared according to manufacturing techniques well known in the pharmaceutical industry and in a variety of dosage forms.

The examples demonstrate the use of quercetin to improve blood pressure and vascular function, thereby, attenuating cardiac hypertrophy and contractile and/or vascular dysfunction.

### EXAMPLE I

To model pressure overload, a surgically induced procedure was used to mimic high blood pressure, known to lead to cardiac hypertrophy. In this procedure the abdominal aorta is constricted to increase the afterload on the heart and produce pressure overload. Male Sprague Dawely 225-250 g rats (n=10) were placed on a diet consisting of 0.15% purified quercetin. Purified quercetin (purchased from Sigma Aldrich) was mixed into standard rat chow at a concentration of 0.15% (1.5 mg quercetin/g of chow). Rats consumed this diet for 7 days.

Given that a rat generally consumes ten grams of chow per 100 g body weight when given free access to the chow, a 250 g rat consumes about 37.5 mg quercetin per day under this regimen. Expressed in pharmacological terms, this is equivalent to a dose of 150 mg quercetin/kg body weight/day.

On the 8th day, 5 rats were surgically modeled for pressure overload by constricting the abdominal aorta to a diameter of 0.63 mm with a Weck hemoclip. The other 5 rats were sham operated (anesthetized and the aorta exposed, but not constricted). The procedure of aortic constriction mimics hypertension, is reproducible, and has been demonstrated in previous studies to induce cardiac hypertrophy after only 2 weeks. Another group of 10 rats were placed on a standard diet with no supplement and treated in an identical fashion with 5 surgically modeled for pressure overload and 5 sham operated. Thus 4 experimental groups were used in this protocol; Sham operated (S) n=5, abdominal aorta constricted (AAC) n=5, Sham operated+quercetin (SQ) n=5, and abdominal aorta constricted+quercetin (AAC-Q) n=5. Two weeks after surgery post-sacrifice morphometric measurements are obtained by measuring total heart weight, left ventricle weight, and calculating heart:body weight and left ventricle:body weight ratios to assess cardiac mass and cardiac hypertrophy. It is found that AAC rats develop significant cardiac hypertrophy compared to sham operated controls. In comparison, AAC-Q rats that were fed a diet supplemented with quercetin have a lower level of cardiac hypertrophy.

In ongoing experiments with greater numbers of rats, we find that quercetin treated rats (AAC-Q) have levels of hypertrophy in between S and AAC rats.

### EXAMPLE II

An identical experimental procedure as described in Example I is used with n=10 rats per group (S, AAC, SQ, AAC-Q) for a total of 40 rats. Surgically induced AAC is again the model for the development of pressure overload and cardiac hypertrophy. Rats subjected to this protocol have very high carotid blood pressure. Rats are anesthetized, a catheter is placed in the caudal and carotid arteries, rats are allowed to regain consciousness for 60 minutes and blood pressures are measured.

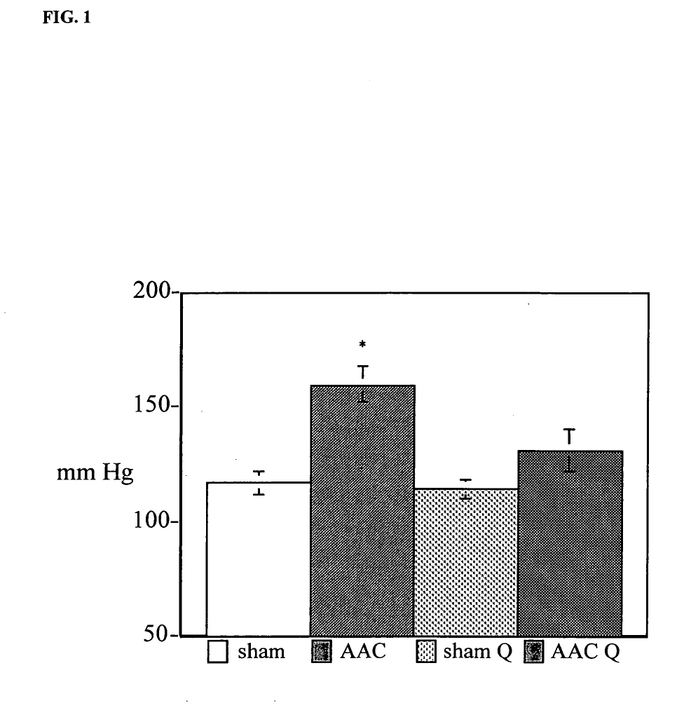

As illustrated in FIG. 1, Quercetin, a dietary flavenoid, reduces blood pressure in the carotid arterial blood pressure in the AAC-Q group. Rats in the AAC-Q group also have attenuated cardiac hypertrophy compared to untreated AAC rats. Therefore, the in vivo data generated with pressure overloaded rats provides information regarding the beneficial effects of quercetin on blood pressure in human subjects.

Mean carotid arterial pressure in AAC rats is approximately 37% greater than controls, however, when AAC rats are supplemented with quercetin (1.5 g/kg of chow), mean carotid arterial pressure is normalized (FIG. 1). Thus, quercetin reduces carotid arterial pressure and consequently reduces the stimulus (pressure overload) for cardiac hypertrophy.

Systolic carotid pressures are significantly reduced in AAC-Q rats compared to AAC (Table 1) (158.8±10.3 vs 201.4±11.4). Diastolic carotid pressures in AAC-Q rats are normalized to the same level of S and SQ (Table 1).

Both systolic and diastolic pressure gradients between caudal and carotid arteries are reduced in AAC-Q rats compared to AAC. Due to the physical constriction of the abdominal aorta, there is a greater pressure in the carotid artery compared to the caudal artery. Thus a pressure gradient exists in AAC rats. AAC-Q rats have reduced systolic pressure gradients and normal diastolic pressure gradients (see Table 1).

Taken together, these data indicate that quercetin supplementation can reduce and/or prevent increases in blood pressure produced by AAC.

TABLE 1
Blood pressure measurements (mm Hg) in caudal and carotid arteries of conscious rats^{1.}

| | Sham | AAC | Sham + Q | AAC + Q | Significance |
|---|---|---|---|---|---|
| Systolic Caudal | 121.2 ± 2.6 | 125.0 ± 5.8 | 114.7 ± 3.2 | 118.3 ± 6.4 | NS |
| Systolic Carotid | 131.1 ± 4.1^{ab} | 201.4 ± 11.4^{c} | 128.2 ± 1.9^{a} | 158.8 ± 10.3^{b} | <0.001 |
| Systolic Gradient | 4.6 ± 2.8^{a} | 71.2 ± 9.6^{c} | 7.3 ± 1.9^{a} | 38.6 ± 5.7^{b} | <0.001 |
| Diastolic Caudal | 105.4 ± 5.5^{b} | 113.7 ± 5.5^{b} | 93.6 ± 3.2^{a} | 104.7 ± 5.5^{b} | 0.049 |
| Diastolic Carotid | 109.9 ± 5.2^{a} | 139.6 ± 7.2^{b} | 106.2 ± 4.8^{a} | 118.4 ± 6.1^{a} | 0.007 |
| Diastolic Gradient | 4.5 ± 2.8^{a} | 220 3.± 1^{b} | 7.0 ± 2.9^{a} | 8.8 ± 2.6^{a} | 0.002 |
| Caudal MAP² | 108.7 ± 2.3 | 117.8 ± 5.6 | 100.6 ± 3.0 | 108.0 ± 7.1 | NS |
| Carotid MAP² | 117.2 ± 4.6^{a} | 160.1 ± 7.9^{b} | 113.5 ± 3.8^{a} | 130.9 ± 9.2^{a} | 0.001 |
| Gradient MAP² | 4.6 ± 2.2^{a} | 37.9 ± 4.7^{c} | 7.2 ± 1.9^{ab} | 18.3 ± 4.4^{b} | <0.001 |

¹Values are means ± standard error of mean. Different letters indicate significant differences at P < 0.05
²MAP = mean arterial pressure.
Sham = sham operated rats, AAC = rats with abdominal aortic constriction, Sham + Q = sham operated rats treated with quercetin, AAC + Q = rats with abdominal aortic constriction treated with quercetin. N = 5 to 10 rats/group.

### EXAMPLE III

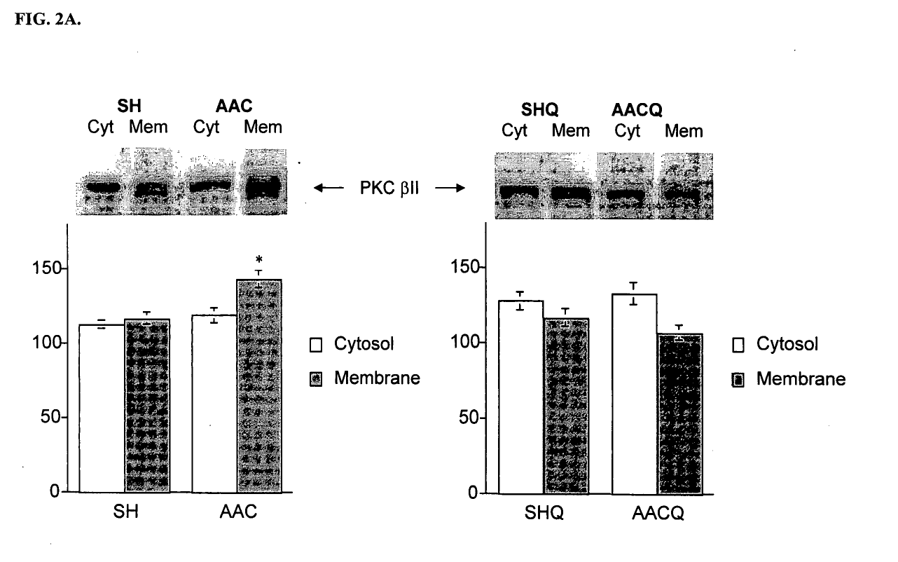

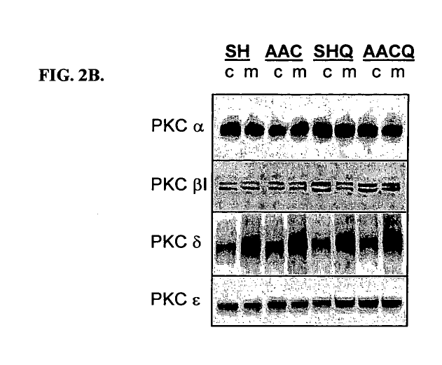

An identical experimental protocol as described in Example I and II was performed. PKC α, βI, βII, ε, δ levels were characterized in each group, as shown in FIG. 2. Following the experimental period, rats were sacrificed, left ventricles were homogenized, and cytosolic & membrane cell fractions isolated using differential centrifugation as previously described (Jalili et al., 1999). Immunobloting using standard protocols was used to asses PKC distribution. Id. Increased abundance in membrane bound fractions is indicative of PKC activation.

Activation of PKC isoforms assessed using Western blots indicated no significant differences in α, βI, ε, or δ isoforms between all groups (see, FIG. 2B). PKC βII translocation and expression was upregulated in AAC and significantly reduced in AACQ. In particular, quercetin supplementation reduced translocation of PKC βII to the membrane fraction (compare AAC with AACQ, as shown in FIG. 2A). Translocation has been identified as a critical mediator of cardiac hypertrophy. Therefore, dietary quercetin may have reduced cardiac hypertrophy during pressure overload and attenuate cardiac hypertrophy. Without wishing to be bound by a theory, the reduction in cardiac hypertrophy may involve the inhibition of PKC βII brought about independently by quercetin supplementation or resulting indirectly from a reduction in blood pressure described in Example II.

PKC blots may be quantified using a UMAX Astra 1220U Scanner, Adobe Photoshop (V 5.0) and NIH Image software (V 1.61). Band density may be measured as scanning pixel units and statistical analyzed using programs such as Sigma Stat software (V 2.01).

### EXAMPLE IV

Using the experimental protocols described in the previous examples with n=7 to 10 per group, the effects of quercetin on vascular reactivity is assessed. Animals are anesthetized, the heart and aorta excised, and vascular reactivity is assessed in segments of aorta using a wire-type myograph.

Post-sacrifice morphometric measurements are obtained by measuring body weight, total heart weight, calculating heart:body weight ratios and heart rate to assess cardiac mass and cardiac hypertrophy. Administration of quercetin reduces cardiac hypertrophy (see, Table 1) and vascular dysfunction associated with cardiac hypertrophy, as demonstrated in experiments described herein.

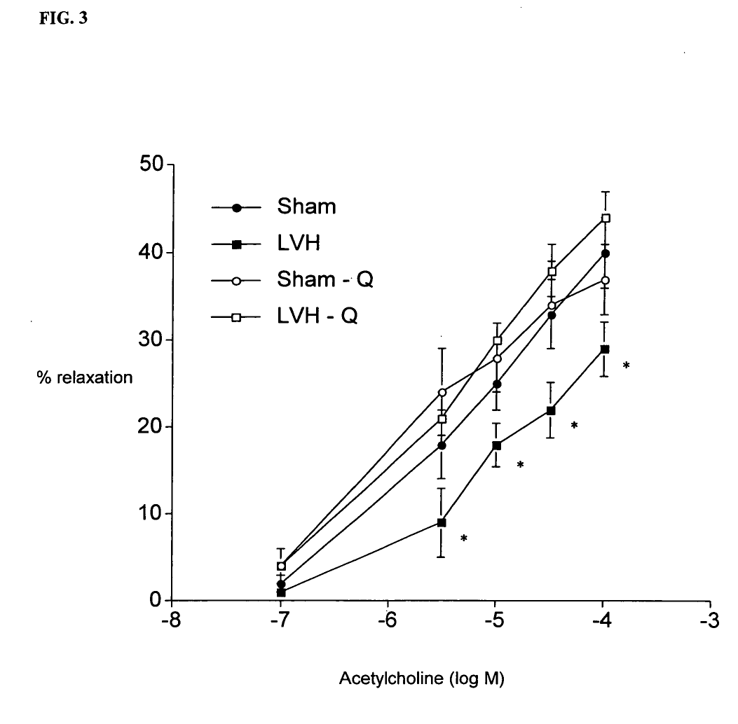

Supplementing quercetin in the diet prevents aortic dysfunction associated with pressure overload and cardiac hypertrophy. AAC-Q rats demonstrate greater endothelium-dependent relaxation in isolated aorta. Therefore, chronic in vivo quercetin-supplementation prevents aortic vascular dysfunction associated with pressure overload in the AAC rat (see, FIG. 3).

As shown in Table 2 and FIG. 3, compared to sham-operated control fed animals (n=10), AAC rats (n=10) had a greater heart weight/body weight ratio (2.93±0.04 vs 3.76±0.18* mg/g), less maximal ACh-evoked relaxation (10⁻⁴ M, 40±4 vs 30±3%*), and less SNP-evoked relaxation (10⁻⁴ M, 71±5 vs 56±3%*) in NE precontracted (10⁻⁵M) aortic segments.

Compared to sham-operated Q-fed animals (n=10), AAC-Q rats (n=9) had a greater heart weight/body weight ratio (2.97±0.06 vs 3.46±0.06*), but similar maximal ACh-evoked relaxation (38±4 vs 44±3%) and SNP-evoked relaxation (71±5 vs 71±4%) in NE precontracted aortic segments (*p<0.05 for all).

Compared to AAC, AAC-Q rats had improved Ach-evoked relaxation (44±3 vs 30±3%*) and improved SNP-evoked relaxation (71±4 vs 56±3%*) (*p<0.05 for all). These data demonstrate that quercetin supplementation reduces blood pressure and prevents aortic dysfunction typically associated with AAC.

TABLE 2
Body weight, organ weights, and levels of quercetin in plasma and liver¹

| | | | | |
|---|---|---|---|---|
| Sham | AAC | Sham + Q | AAC + Q | Significance |

| | | | | | |
|---|---|---|---|---|---|
| Body wt. (g) | 335 ± 7 | 321 ± 6 | 342 ± 10 | 342 ± 7 | NS |
| Heart wt. (g) | 0.98 ± 0.02^{a} | 124 ± 0.06^{b} | 1.01 ± 0.03^{a} | 1.18 ± 0.03^{b} | <0.001 |
| Heart (mg):body wt (g) | 2.93 ± 0.04^{a} | 3.76 ± 0.18^{c} | 2.97 ± 0.06^{a} | 3.46 ± 0.06^{b} | <0.001 |
| Heart Rate (BPM) | 382 ± 16^{a} | 410 ± 8^{b} | 402 ± 9^{b} | 414 ± 9^{b} | 0.002 |
| Lung wt. (g) | 1.46 ± 0.05 | 1.45 ± 0.07 | 1.50 ± 0.05 | 1.48 ± 0.05 | NS |
| Liver wt. (g) | 12.3 ± 0.5 | 12.1 ± 0.5 | 11.1 ± 0.7 | 10.6 ± 0.6 | NS |
| Plasma quercetin (μg/mL)² | 0^{a} | 0^{a} | 326 ± 0.11^{b} | 396 ± 0.89^{b} | <0.001 |
| Liver quercetin (μg/mL)³ | 0 | 0^{a} | 3.00 ± 0.47^{b} | 352 ± 089^{b} | <0.001 |

¹Values are means ± standard error of mean. Different letters indicate significant differences at P < 0.05
²Plasma quercetin levels composed of free and conjugated quercetin.
³Liver quercetin levels composed of free quercetin, free and conjugated O-methoxy quercetin
Sham = sham operated rats (n = 10), AAC = rats with abdominal aortic constriction (n = 10), Sham + Q = sham operated rats treated with quercetin (n = 10), AAC + Q = rats with abdominal aortic constriction treated with quercetin (n = 9).

In this model of LVH, Q-supplementation limits aortic vascular dysfunction. In addition, quercetin treatment significantly reduces, blood pressure, PKC βII activation, and cardiac hypertrophy.

### EXAMPLE V

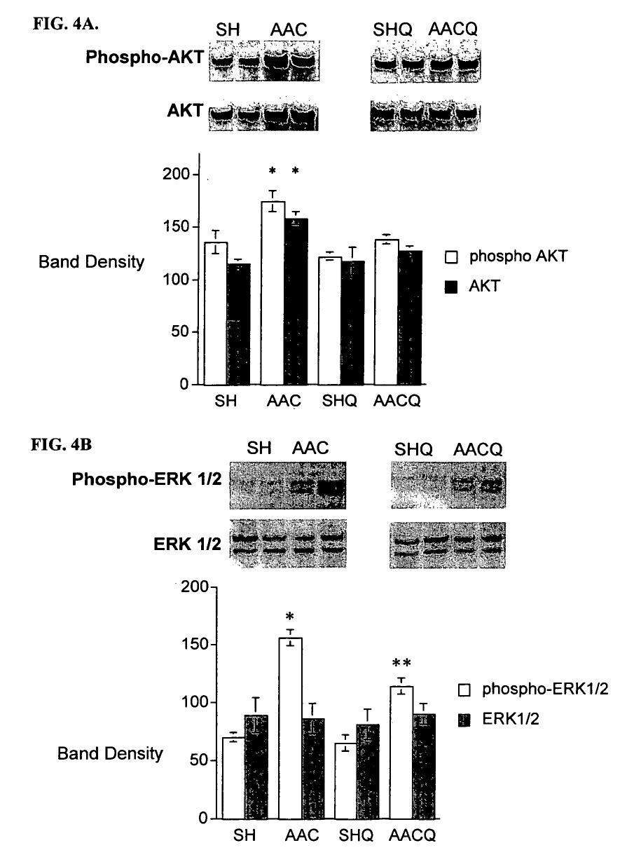

Determination of ERK1/2 and Akt signaling status (FIG. 4). All extraction procedures were performed at 4° C. Left ventricles from Sham (n=5), AAC (n=5), Sham+quercetin (n=5), and AAC+quercetin (n=5) were homogenized with a tissuemizer in 1 ml of ice-cold RIPA buffer (50 mM Tris-HCl (pH 7.4), 150 mM NaCl, 1% Nonidet P-40, 0.25% sodium deoxycholate, 1 mM sodium orthovanadate, 1 mM NaF, and 10 μl/mL Sigma protease inhibitor cocktail (Sigma, St. Louis, Mo., cat. #P-8340)). After homogenization, the samples were sonicated twice on ice and centrifuged at 11,000 X g for 10 min. at 4° C. Supernatants were recovered and stored at −80° C. for subsequent immunoblotting. Protein concentration of whole heart cell lysate was determined using a Bio-Rad Protein assay (Bio-Rad, Hercules, Calif.) with bovine serum albumin (BSA) as a standard.

Western blotting, transfer and densitometry: Electrophoresis and transfer of proteins to PVDF membranes were done as previously described (18, 19). Primary antibody directed against PKC α, βI, βII, δ, and ε (Santa Cruz Biotechnology, Santa Cruz, Calif.) were incubated overnight at 4° C. in a 1:1000 dilution. Antibodies directed against phospho ERK 1/2, phospho JNK, ph c-Raf (Cell Signal Technology, Beverly, Mass.), and phospho PKC β (Santa Cruz Biotechnology, Santa Cruz, Calif.), were incubated at a 1:1000 dilution for 48 h at 4° C. in 5% BSA Tris buffer with 0.05% Tween-20. Secondary antibody conjugated to horseradish peroxidase (goat anti rabbit, (Cell Signal Technology, Beverly, Mass.)) was incubated for 1 h at 1:10,000 dilution. Signals were visualized by enhanced chemiluminescence (Cell Signal Technology, Beverly, Mass.). Relative band density of immunoblots on film were measured with a scanner using NIH 1.63 image software (National Institutes of Health, Rockville, Md.).

### EXAMPLE VI

Left Ventricular Hypertrophy (LVH) in a subject may be diagnosed using echocardiography (ECG) techniques (Am Heart J, 1949;37: 161; Circulation, 1987;3: 565-72; Circulation, 1990; 81:815-820; and Am Heart J, 1986:75:752-58). A subject meeting the criteria is highly likely to suffer from LVH. The general ECG criteria used to diagnose LVH include, a increased QRS amplitude (voltage criteria; i.e., tall R-waves in LV leads, deep S-waves in RV leads), delayed intrinsicoid deflection in V6 (i.e., time from QRS onset to peak R is ≧0.05 sec), widened QRS/T angle (i.e., left ventricular strain pattern, or ST-T oriented opposite to QRS direction), and a leftward shift in the frontal plane QRS axis (Lipman B, Cascio T. ECG Assessment and Interpretation. Philadelphia, Pa.:FA Davis Co.; 1994).

### EXAMPLE VII

The spontaneously hypertensive (SH) rat is used to demonstrate the effectiveness of querceten, since this strain is well documented to have normal blood pressure until 5-6 weeks of age (Makita and Yasuda, 1990). After 5-6 weeks, blood pressure steadily rises until reaching a peak systolic pressure of 200-220 mm Hg at 11 weeks of age. This etiology is similar to humans predisposed to hypertension that exhibit rising blood pressure with age. As a result of developing hypertension, SH rat also develops cardiac hypertrophy by 8 weeks of age (Rizzoni et al., 1994). Therefore, the SH rat has been recognized as an ideal model to test suitability of blood pressure lowering agents (Roba, 1976).

Data regarding the effectiveness of quercetin in the rat studies may be analyzed using a one way ANOVA (SPSS v. 10) with LSD post hoc test used to detect significant differences between groups.

Male weanling WKY and SH rats (4 weeks of age) (purchased from Harlan, Indianapolis, Ind.) are measured for baseline blood pressures and body weights and randomly divided into dietary groups; Group 1; WKY normotensive+control diet, Group 2; SH rat+control diet, Group 3; SH rat+1.5 g quercetin/kg diet. After 5 and 10 weeks of diet treatment blood pressure, echocardiograms and body weight are measured. Animals are sacrificed after the 10^{th} week of diet treatment and coronary arteries are removed to determine endothelial function and nitric oxide production.

### EXAMPLE VIII

The SH rat (SHR) was used to demonstrate the effectiveness of quercetin (20). As described herein, this strain is well documented to have normal blood pressure until about 5-6 weeks of age (Makita and Yasuda, 1990). After about 5-6 weeks, the rats suffer from steadily increasing blood pressure from about 6 weeks of age until peaking at about 12-15 weeks of age.

Blood pressure was measured in SHR and SHRQ rats by lightly anesthetizing the animals using ketamine/xylazine (75/3.75 mg/kg i.p.). Rats were prewarmed for 15 minutes on heated platform and blood pressure was measured using a Visitech BP 2000 blood pressure system. Pressure measurements are reported as the average of between 4 and 8 separate measurements taken over a 70 second period. All rats fully recovered within 30 after receiving anesthesia.

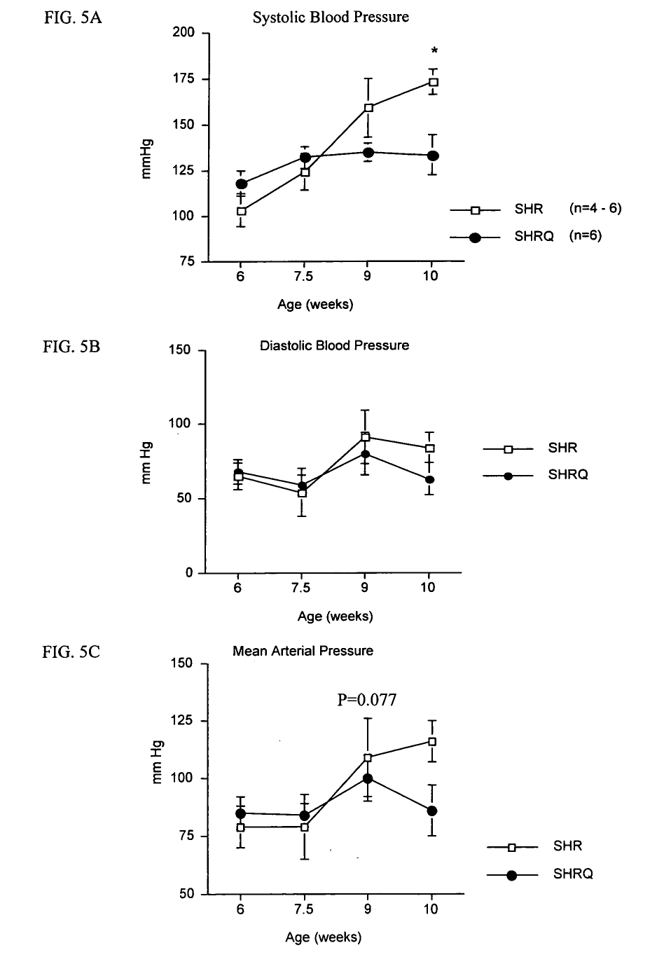

Male SH rats (4 weeks of age) (purchased from Harlan, Indianapolis, Ind.) were measured for baseline blood pressures and body weights and randomly divided into dietary groups; SH rat+control diet (SHR) (n=4 to 6), and SH rat+1.5 g quercetin/kg diet (SHRQ) (n=6) (see, FIGS. 5A and 5C). Blood pressure blood pressure was measured for each group at 6, 7.5, 9 and 10 weeks. Dietary supplement was initiated at 5 weeks and maintained throughout the experiment. This experiment demonstrates that quercetin delayed the onset of hypertension in this genetic model of hypertension.

In a separate experiment using SH rats (dietary supplement was likewise initiated at 5 weeks), blood pressure was measured at 17 weeks. No significant difference in blood pressure was observed between control and quercetin treated rats at 17 weeks. However, it is believed that by 17 weeks the severity of the phenotype in this genetic model system masks the effect of querceten treatment.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

### REFERENCES

All references, including publications, patents, and patent applications, cited herein are hereby incorporated by reference to the same extent as if each reference were individually and specifically indicated to be incorporated by reference and were set forth in its entirety herein. The following list of references is hereby incorporated by reference in their entirety:

1. Melina D, Coliricci F, Buerra G, Melina G, Frustaci A, Caldarulo M, Guerrera C. Prevalence of left ventricular hypertrophy and cardiac arrythmias in borderline hypertension. American Journal of Hypertension 5(8):570-573, 1992;

2. Brody S, Preut R, Schommer K, Schurmeyer T H. A randomized controlled trial of high dose ascorbic acid for reduction of blood pressure, cortisol, and subjective responses to psychological stress. Psychopharmacology. 2002;159:319-24;

3. Aviram M, Dornfeld L. Pomegranate juice consumption inhibits serum angiotensin converting enzyme activity and reduces systolic blood pressure. Atherosclerosis. 2001;158:195-8;

4. Koba K, Abe K, Ikeda I, Sugano M. Effects of alpha-tocopherol and tocotrienols on blood pressure and linoleic acid metabolism in the spontaneously hypertensive rat (SHR). Biosci Biotechnol Biochem. 1992;56:1420-3;

5. Duarte J, Perez-Palencia R, Vargas F, Ocete M A, Perez-Vizcaino F, Zarzuelo A, Tamargo J. Antihypertensive effects of the flavonoid quercetin in spontaneously hypertensive rats. Br J. Pharmacol. 2001; 133:117-24;

6. Graefe EU, Wittig J, Mueller S, Riethling A K, Uehleke B, Drewelow B, Pforte H, Jacobasch G, Derendorf H, Veit M. Pharmacokinetics and bioavailability of quercetin glycosides in humans. J Clin Pharmacol. 2001;41:492-9;

7. Makita N, Yasuda H. Alterations of phosphoinositide-specific phospholipase C and protein kinase C in the myocardium of spontaneously hypertensive rats. Basic Res Cardiol. 1990;85:435-43;

8. Rizzoni D, Castellano M, Porteri E, Bettoni G, Muiesan M L, Agabiti-Rosei E. Vascular structural and functional alterations before and after the development of hypertension in SHR. Am J Hypertens. 1994;7:193-200;

9. Roba J L. The use of spontaneously hypertensive rats for the study of anti-hypertensive agents. Lab Anim Sci. 1976;26:305-19;

10. Pedro-Botet J, Covas M I, Martin S, Rubies-Prat J. Decreased endogenous antioxidant enzymatic status in essential hypertension. J Hum Hypertens. 2000;14:343-5.

11. Vaziri N D, Wang X Q, Oveisi F, Rad B. Induction of oxidative stress by glutathione depletion causes severe hypertension in normal rats. Hypertension. 2000;36: 142-6;

12. Vaziri N D, Liang K, Ding Y. Increased nitric oxide inactivation by reactive oxygen species in lead-induced hypertension. Kidney Int. 1999;56:1492-8;

13. Newaz M A, Nawal N N. Effect of gamma-tocotrienol on blood pressure, lipid peroxidation and total antioxidant status in spontaneously hypertensive rats (SHR). Clin Exp Hypertens. 1999;21:1297-313;

14. Galley H F, Thornton J, Howdle P D, Walker BE, Webster NR. Combination oral antioxidant supplementation reduces blood pressure. Clin Sci (Colch). 1997;92:361-5;

15. Vaziri N D, Ni Z, Oveisi F, Trnavsky-Hobbs D L. Effect of antioxidant therapy on blood pressure and NO synthase expression in hypertensive rats. Hypertension. 2000;36:957-64;

16. Naguib Y M. A fluorometric method for measurement of oxygen radical-scavenging activity of water-soluble antioxidants. Anal Biochem. 2000;284:93-8;

17. Jalili T, Takeishi Y, Song G, Ball N A, Howles G, Walsh R A. PKC translocation without changes in Galphaq and PLC-beta protein abundance in cardiac hypertrophy and failure. Am J Physiol. 1999;277:H2298-304;

18. Jalili, T., Y. Takeishi, G. Song, N. A. Ball, G. Howles, and R. A. Walsh. 1999. PKC translocation without changes in Galphaq and PLC-beta protein abundance in cardiac hypertrophy and failure, Am J Physiol 277:H2298-2304;

19. Takeishi, Y., Q. Huang, J. Abe, M. Glassman, W. Che, J. D. Lee, H. Kawakatsu, E. G. Lawrence, B. D. Hoit, B. C. Berk, and R. A. Walsh. (2001) Src and multiple MAP kinase activation in cardiac hypertrophy and congestive heart failure under chronic pressure-overload: comparison with acute mechanical stretch, J Mol Cell Cardiol 33:1637-48; and

20. OKAMOTO, K. and AOKI, K. (1963). Development of a strain of spontaneously hypertensive rats, Jap. Circ. J. 27:282-293.

We claim:

1. A method for preventing or delaying the onset of or slowing the progression of hypertension in a subject, said method comprising:
administering to a subject a nutritional supplement including quercetin, wherein said supplement is administered in an amount effective in preventing, delaying or slowing the onset or progression of hypertension.

2. The method according to claim 1, wherein an amount effective in preventing, delaying or slowing the onset or progression of hypertension comprises a daily dosage of quercetin of about 15 mg to about 250 mg/kg of body weight.

3. The method according to claim 1, wherein said quercetin is present in an amount to provide a daily dosage of about 0.1 g to about 17.5 g.

4. The method according to claim 1, wherein said supplement additionally contains other nutrients selected from the group consisting of vitamin B₁₂, vitamin B₆, non-flavonoid antioxidants, minerals and trace metals.

5. The method according to claim 1, wherein said supplement additionally contains a non-flavonoid antioxidants selected from the group consisting of selenium, vitamin E, vitamin C, niacin, beta-carotene and coenzyme Q10, or combinations thereof.

6. The method according to claim 5, wherein said supplement additionally contains a non-flavonoid mineral or trace metal selected from the group consisting of zinc, copper, magnesium, manganese, chromium, molybdenum, iron and calcium.

7. The method according to claim 1, wherein said supplement further includes one or more agents selected from the group consisting of a taste-improving agent, coloring agent, preservative, stabilizer, regulator and emulsifier.

8. The method according to claim 1, wherein said nutritional supplement is administered orally as a liquid.

9. The method according to claim 1, wherein said supplement is administered orally as a nutritional food.

10. The method according to claim 1, wherein said hypertension is an agent in the development of left ventricular hypertrophy.

11. The method according to claim 8, wherein said supplement additionally contains vitamin C or vitamin E.

12. The method according to claim 9, wherein said supplement additionally contains vitamin C or vitamin E.

13. The method according to claim 9, wherein said supplement is an energy bar.

14. The method according to claim 8, wherein said supplement is a beverage.

15. The method according to claim 1, wherein said supplement is administered in an amount effective in delaying the onset of hypertension.

16. The method according to claim 1, said method further comprising determining if a subject is suffering from hypertension or is prone to the development of hypertension.

17. The method according to claim 14, wherein said beverage is orange juice.

18. A nutritional supplement useful in aiding in preventing, delaying the onset of and/or slowing the progression of hypertension, said nutritional supplement comprising:
quercetin or a quercetin glycoside in an amount which provides a daily dosage of about 0.1 g to about 17.5 g per day;
a source of fat;
a source of carbohydrate; and
a source of protein, thereby providing a nutritional supplement which aids in preventing, delaying or slowing the onset or progression of hypertension.

19. The supplement of claim 18, wherein said supplement is formulated as a food or beverage.

20. The supplement of claim 19, wherein the supplement additionally contains another nutrient selected from the group consisting of vitamin B₁₂, vitamin B₆, non-flavonoid antioxidants, minerals and trace metals.

21. The supplement of claim 19, wherein said supplement additionally contains a antioxidant selected from the group consisting of selenium, vitamin E, vitamin C, niacin, beta-carotene and coenzyme Q10.

22. The supplement of claim 18, wherein said supplement additionally contains one or more compound selected from the group consisting of zinc, copper, magnesium, manganese, chromium, molybdenum, iron and calcium, or combinations thereof.

23. The supplement of claim 18, wherein said supplement further includes one or more taste-improving agent, coloring agent, preservative, stabilizer, regulator, emulsifier, or mixtures thereof.

24. The nutritional supplement of claim 20, wherein said nutritional bar further comprises a dietary fiber selected from the group consisting of soluble fiber, insoluble fiber, fermentable fiber, non-fermentable fiber and mixtures thereof.

25. The nutritional supplement of claim 19, where said nutritional supplement is in the form of a cookie.
